(19) United States
(12) Patent Application Publication
(10) Pub. No.: US 2010/0035350 A1
(43) Pub. Date: Feb. 11, 2010

(54) DEVICE AND METHOD FOR LABELING AND MEASURING THE RADIOCHEMICAL PURITY OF RADIO-DRUGS

(75) Inventors: Arturo Guillermo Casale, Buenos Aires (AR); Jorge Osvaldo Nicolini, Buenos Aires (AR); Elvira Calvo Kock, New York, NY (US)

Correspondence Address: EGBERT LAW OFFICES, 412 MAIN STREET, 7TH FLOOR, HOUSTON, TX, 77002, US

(73) Assignee: ARCANA INTERNATIONAL, INC, New York, NY (US)

(21) Appl. No.: 12/523,318

(22) PCT Filed: Jan. 20, 2007
(86) PCT No.: PCT/US07/60812

§ 371 Date: Jul. 15, 2009

(51) Int. Cl. G01N 23/00; G01N 33/00

(52) U.S. Cl. 436/57; 422/68.1

(57) ABSTRACT

The present invention relates to a device for labeling and measuring the radio-chemical purity of a radio-drug product. There is a microprocessor (10) that is the core of the equipment, having a PC connection (11) and also a display (9) to send messages to the operator, and detectors (8) for the control operation of the radio-chemical purity and the regulated heater. The present invention also includes a partition vessel (7), a labeling vessel (12), and a regulated heater (130 that complete the set that defines the purposed device. The control is performed by the microprocessor (10) to accurately determine the temperature at which the product is labeled and the time during which it will be subject to the temperature. Also, the device allows measurement, in a few seconds, of the radio-chemical purity of the labeled products by introducing a drop of the already labeled product in a partition vessel (7).

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a device to be used in nuclear medicine services and/or radio-pharmacies to aid the professional technician and/or physician in making radio-pharmaceutical labeling and radio-chemical purity controls on the labeled radio-drug before it is injected in the body of a patient. More specifically, the present invention relates to a device for labeling and measuring the radio-chemical purity of labeled radio-drugs, such as sestamibi (MIBI)-Tc-99m, bicisate (ECD)-Tc-99m and HMPAO-Tc-99m, although it maybe used for any kind of radio-pharmaceutical labeling and controls.

2. Description of Related Art Including Information Disclosed Under 37 CFR 1.97 and 37 CFR 1.98

The difficulties encountered when labeling radio-drugs used in nuclear medicine studies are well known. In the case of MIBI, the mixture of MIBI and eluate from the technetium-99-metastable (Tc-99m) generator has to be subjected to a temperature of 100° C. for 10 minutes. The recommended method is a water bath (boiling water). At present, there is no economical and simple device that may offer a fast, simple, and secure operation, for speed and practical reasons. Thus, professionals usually introduce the product into a microwave oven and heat it for a few seconds. As there is obviously no way of accurately checking the temperature of the product with this heating method, the product is usually over-heated, which may result in wrong labeling, decomposition of the labeled product, as well as time waste, loss of radioactive material, and unnecessary irradiation of the staff. Unfortunately, if the product is not correctly labeled and its purity is not checked, the patient is subject to unnecessary irradiation and the study result is incorrect, so the operation has to be repeated, with the afore-mentioned consequences.

Ensuring the correct labeling of the product and checking its radio-chemical purity before initiating the injection procedure is therefore of utmost importance.

There are commercially available devices that help the professional in ensuring a correct labeling. The main problem is that these devices consist of a complicated and expensive apparatus using the “Peltier’ method for heating the product but has no checking systems for controlling the labeling process.

### BRIEF SUMMARY OF THE INVENTION

The present invention relates to a device for labeling and measuring the radio-chemical purity of radio-drugs, comprising a regulated and armored heating unit into which the vessel containing the product to be labeled, for example MIBI with Tc-99m eluate, is inserted. The vial has the ability to automatically control the temperature, as well as to label time. The control is performed by a microprocessor that forms an integral part of the apparatus and allows the professional to accurately determine the temperature at which the product is labeled and the time during which it will be subject to the temperature. Also, the device proposed allows measurement, in a few seconds and without the need of complex systems such as radio-chromatographs, of the radio-chemical purity of the labeled products by introducing a drop of the already labeled product in a partition vessel. The device is provided with collimated detectors on each phase which, within a few seconds, detect the labeling percentage versus its partition in a system of two immiscible solvents chosen for such purpose, for example, chloroform and water. The device measures radioactivity in each phase and through calculation obtains the labeling percentage. If the statistical value is low, it will send a message to the operator indicating “Low Count”, so the measuring time should be increased or more samples should be collected.

If the percentage is higher than 90%, the apparatus will send a “Labeling OK” message, and if the percentage is lower than 90% it will send a “Wrong Labeling, Repeat Process” message, since the percentage accepted by the pharmacopoeia is higher than or equal to 90% of radio-chemical purity.

Accordingly, by means of an operation lasting only a few seconds and through a simple manipulation of the elements, the professional may perform the labeling operation of the afore-mentioned radio-drugs in a secure way.

Summing up, the present invention is referred to a device for labeling and measuring the radio-chemical purity of radio-drugs, comprising an armored heating unit controlled by a microprocessor capable of performing the radio-drug labeling operation, and a detector assembly also connected to the microprocessor capable of determining the labeling percentage and consequently the radio-chemical purity of the product. It is also referred to a method for measuring the radio-chemical purity of radio-drugs, comprising the stages of:
allowing the radio-drug to cool down and transferring one drop to a partition vessel using a 1-ml syringe, once the product has been labeled;
shaking and placing the vessel in the measuring device; and
pressing the “Purity” button and, within a few seconds, calculating, by means of the microprocessor, the labeling percentage.

The result is shown on the display. If the statistical value is low, the message on the display will indicate “Low Statistical Value, Increase Measuring Time or Add More Activity to Partition Vessel”, and so the measuring time should be increased. If the percentage is higher than or equal to 90%, then the Operator will see a message indicating “LABELING OK”. On the contrary, if the percentage is lower than 90%, the message will indicate “WRONG LABELING—REPEAT PROCESS”.

The present invention also refers to a method for labeling and measuring the radio-chemical purity of radio-drugs, comprising the stages of:
introducing the vial containing the radio-drug to be labeled and the Tc-99m eluate inside the armored heater and, after configuring the system, labeling the radio-drug; and
indicating, once the labeling time set has elapsed, by means of a buzzer, the end of the labeling operation.

### BRIEF DESCRIPTION OF THE SEVERAL VIEWS OF THE DRAWINGS

FIG. 1 illustrates a perspective view of the present invention.

FIG. 2 is a schematic view showing, in simple visual terms, the inner structure of the device proposed.

FIG. 3 is a schematic view of a partition vessel 14 for purity control.

### DETAILED DESCRIPTION OF THE INVENTION

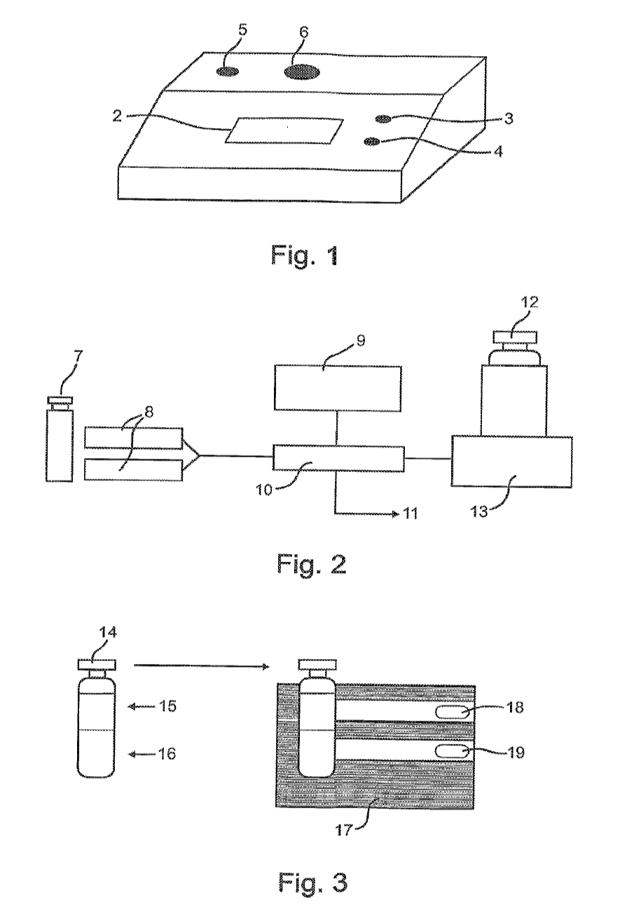

FIG. 1 illustrates a perspective view of the device proposed, on whose top the opening for introducing the vial containing the product to be labeled 6 may be clearly seen. Inside the receptacle, there is a heater regulated and controlled by a microprocessor forming an integral part of the apparatus. On the left side, there is an opening for the partition vessel 5, where a drop of the product should be introduced to control the radio-chemical purity after labeling.

FIG. 2 is a schematic view showing, in simple visual terms, the inner structure of the device proposed. It comprises a microprocessor 10 that is the core of the equipment, having a PC connection 11 and also a display 9 to send messages to the operator, detectors 8 for the control operation of the radio-chemical purity and the regulated heater. It can clearly be seen to include the partition vessel 7, a labeling vessel 12, and a regulated heater 13 that complete the set that defines the purposed device.

Finally, FIG. 3 is a schematic view of a partition vessel 14 for purity control including an aqueous phase 15 and an organic phase 16. A collimating lead piece 17 includes an aqueous phase detector 18 and an organic phase detector 19.

The device proposed, designated with general reference number 1, comprises an outer shell including a display 2 in the center of its upper side, and two “Labeling” 3 and “Purity” 4 controls next to it. On the left upper sector, there is an orifice for placing the partition vessel 5, and next to it there is an orifice 6 for placing the vial containing the radio-drug to be labeled. The radio-drug to be labeled may be MIBI(sestamibi) and tetraphosmine, for cardiologic and tumoral studies; ECD (bicisate) and HMPAO for cerebral studies to be labeled with the Tc-99m generator eluate.

The device has 2 defined functions:
1. Labeling;
2. Purity Measurement and a configuration page.

1. Labeling

The labeling function consists of a heater controlled by a microprocessor at the selected temperature; for example, for MIBI labeling a temperature of 100° C. is selected. The heating time is also selected based on the desired labeling type. For example, for MIBI a heating time of 10 minutes is selected. This selection is performed through the buttons the device has on top through a simple interactive menu. Once selected, the parameters are recorded for the following labeling operation.

The radio-drug is mixed with the Tc-99m generator eluate and is introduced into the heater. The heating process is started by pressing a button designated for such a purpose.

The heater initiates its thermal excursion based on a mathematical algorithm that provides more power as the temperature difference between the temperature set and the one measured by the sensor increases. When the difference between both temperatures is 0° C., the heating power is zero, and it reaches its maximum when the thermal difference is 40° C. or higher. In this way, a fast thermal increase up to the desired temperature is achieved, with a high stabilization at the desired temperature.

Algorithm Description:

TD: Temperature difference between the desired temperature and the temperature set.

If TD>40° C., then the heating power is=100% (maximum)

If TD>35° C., then the heating power is=80%

If TD>30° C., then the heating power is=70%

If TD>25° C., then the heating power=60%

If TD>20° C., then the heating power is=50%

If TD>15° C., then the heating power is=40%

If TD>10° C., then the heating power is=20%

If TD>5° C., then the heating power is=10%

If TD=0° C. or negative, then the heating power is=0% (null)

Once the labeling time has elapsed, the heater is turned off and a buzzer sounds indicating that the product is ready.

2. Purity Measurement

After the labeling process, a drop from the labeling vessel should be extracted with a 1-ml syringe and it should be introduced in the partition vessel.

The partition vessel for purity checking consists in a small 5-ml vial containing 1.5 ml of chloroform or another immiscible organic solvent and 1.5 ml of water or physiological solution or another solvent that is immiscible in the previous one (see FIG. 4). A drop of the labeled product is introduced in this vessel and it is shaken for radioactivity partition. The labeled radio-drug will be extracted by the organic phase, whereas the free technetium-99m will be extracted by the aqueous phase. The radio-chemical purity is determined by measuring radioactivity of each phase and it is reported as a labeling percentage according to the following equation:

% L=Labeling percentage

Cop=Counts detected in the organic phase

Cap=Counts detected in the aqueous phase

Cf=Cross linking Factor

Equation

% L=(Cop−Cap*Cf)*100/((Cop−Cap*Cf)+(Cap−Cop*Cf))

The cross linking factor is a correction introduced to compensate for the lack of absolute collimation and Compton's effect of radiation dispersion due to the collimator material.

The measuring time does not directly influence the equation, but it does influence its statistical values. The program detects if the statistical values are low through the addition of total counts:

Total Counts=Cop+Cap

The report ends with an indication in % L, but if Total Counts is less than or equal to 20, the indication is: “Low Statistics, Increase Measuring Time or Add More Activity to Partition Vessel”.

If Total Counts is higher than 20, % L is reported on the display and if it is higher than or equal to 90% it indicates: “Labeling OK, Radio-drug May be Injected”; if % L is lower than 90%, it indicates: “Wrong Labeling, Repeat Procedure” and that preparation should be discarded.

In the inside, the apparatus includes a power supply for the detectors, a heater and electronic logic with its security fuse and ground connection. In the case of Geiger Mueller detectors, the power supply provides 500 V regulated with a 5-megohm current limiting resistor, which makes the outbound current very low, in the order of nanoamperes, in order to avoid electric clashes. For the heater, the power supply provides 12 V of alternating current with an amperage of up to 2 so as to reach a power of up to 24 watts. For logic circuits, the power supply provides 5 regulated volts.

The apparatus also includes a motherboard including a microprocessor of the PIC 16FXXX kind with memory and peripheral electronics comprised of:
a. Operational amplifiers of the pulses from the detectors;
b. Optoinsulators for electrical isolation of pulses between the detectors and the microprocessor;
c. Operational amplifier of the temperature sensor;
d. Heater control relay;
e. Communication circuit for RS232 PC consistent in MAX 232; and
f. Buzzer for sound indication.

The apparatus further includes an alphanumeric double-line display.

There is a 12-volt, 20-watt clamp-type heater, armored, with temperature sensor.

There is also a collimated detectors towards the partition portions consisting in two Geiger Mueller tubes or semiconductor-type detectors (for example, Hamamatsu) placed inside a mechanized lead piece in such a way that each detector “observes” only the radiation from one of the phases of the liquid/liquid partition vessel, as observed in FIG. 3 that illustrates a Partition Vessel for Purity Control and Collimating Lead Piece.

The bottom the apparatus is provided with a serial output or similar for a PC connection, with the aim of recording the process and issuing a report about it. The report is useful for clinical research protocols or for recording the labeling operations performed. The report includes the following:
Labeling Day/Time;
Name of the Technician/Professional in charge of the labeling operation;
Laboratory supplying the radio-drug;
Radio-drug lot number;
Laboratory supplying the Tc-99m generator;
Tc-99m generator lot number;
Activity used in labeling;
Labeling percentage obtained; and
Remarks.

Configuration Page: It may be accessed by pressing both buttons. The user may set the following parameters:
Heater Temperature;
Labeling Time; and
Purity Measurement Time.

There are advantages provided by the device.

It eliminates the use of microwave ovens for labeling, which are currently used, since heating radio-drugs at uncontrolled temperatures is not recommended due to uncertain results for both the operator and the patient.

It avoids vial explosion inside the microwave oven, as often happens in practice.

It avoids another commonly used procedure for labeling, which is the water bath, that is, the use of boiling water to label the product.

The heater used by the device proposed is controlled by an electronic sensor integrated by a mathematical algorithm through microprocessor 9, indicating the end of the process by sound means, which facilitates the technical operator's task since he/she does not need to check the labeling time nor temperature.

Measuring the radio-chemical purity is ESSENTIAL and is achieved by a fast and simple method, whose result is reported within a few seconds. This control avoids wrong medical results, an unnecessary waiting time for the patient, and the repetition of the study with the consequent additional irradiation of the patient.

It is designed to be used inside the heated laboratory in an autonomous way.

It may be connected to a PC through a regular serial cable for printing the labeling reports for research protocols.

As was already explained above, the present invention also encompasses a method for measuring the purity of radio-drugs, comprising a solvent partition system and an assembly of two collimated radiation detectors towards each of the liquid phases, allows determining the radio-chemical purity in order to determine if the radio-drug may be injected to the patient.

The above mentioned method also includes the stages of:
introducing the vial containing the radio-drug to be labeled and the Tc-99m eluate inside the armored heater and, after configuring the system, labeling the radio-drug; and
indicating, once the labeling time set has elapsed, by means of a buzzer, the end of the labeling operation.

Additionally, the purposed method comprises the stages of:
allowing, once the product has been labeled, the radio-drug to be cooled down and one drop is transferred to a partition vessel using a 1 -ml syringe;
shaking and placing the in the measuring device; and
pressing the “Purity” and within a few seconds the device calculates, by means of the microprocessor, the labeling percentage.

The result is shown on the display; if the statistical value is low, the message on the display will indicate “Low Statistical Value, Increase Measuring Time or Add More Activity to Partition Vessel”, and so the measuring time should be increased; if the percentage is higher than or equal to 90%, then the Operator will see a message indicating “LABELING OK”; on the contrary, if the percentage is lower than 90%, the message will indicate “WRONG LABELING—REPEAT PROCESS”. A PC connection controls and records the labeling operations on the PC.

Additionally, the purposed method for measuring the radio-chemical purity of radio-drugs is specifically designed for labeling MIBI with Tc-99m, and for labeling control (radio-chemical purity) but which also allows to:
label and control ECD;
label and control HMPAO;
label and control Tetraphosmine;
label MAG3; and
by changing the partition system, allowing the Examiner to control the whole range of radio-drugs.

We claim:

1. A device for labeling and measuring the radiochemical purity of radio-drugs, said device comprising:
an armored heating unit controlled by a microprocessor capable of performing the radio-drug labeling operation; and
a detector assembly connected to said microprocessor capable of determining the labeling percentage and consequently the radio-chemical purity of the product.

2. The device for labeling and measuring the radiochemical purity of radio-drugs, according to claim 1, further comprising:
an outer shell having a display, two buttons labeled as “Labeling” and “Purity”, an orifice for placing a partition vessel inside, and a heater for placing the vial containing the product to be labeled.

3. A method for measuring the radiochemical purity of radio-drugs, said method comprising the steps of:
using a solvent partition system; and
using an assembly of two collimated radiation detectors towards each of the liquid phases, determining radio-chemical purity in order to determine if the radio-drug may be injected to a patient.

4. A method for measuring the radiochemical purity of radio-drugs, said method comprising the stages of:
introducing a vial containing a radio-drug to be labeled and a Tc-99m eluate inside an armored heater;
labeling the radio-drug after configuring the system; and
indicating, once labeling time set has elapsed, by a buzzer, an end of said labeling operation.

5. A method for measuring radio-chemical purity of radio-drugs, comprising the steps of:
allowing the radio-drug to cool down, once product has been labeled, one drop being transferred to a partition vessel using a 1-ml syringe;
shaking and placing the vessel in the measuring device;
pressing the “Purity” button and within a few seconds, calculating the labeling percentage by the microprocessor; and
showing the result on the display; wherein, if the statistical value is low, then the message on the display indicates “Low Statistical Value, Increase Measuring Time or Add More Activity to Partition Vessel”, the measuring time being increased; and wherein, if the percentage is higher than or equal to 90%, then the message indicates “LABELING OK”; and wherein, if the percentage is lower than 90%, the message indicates “WRONG LABELING—REPEAT PROCESS”.

6. The method for measuring the radio-chemical purity of radio-drugs, in accordance with claim 5, said microprocessor is a PC connection, controlling and recording labeling operations through a PC.

7. The method for measuring the radio-chemical purity of radio-drugs, in accordance with claim 5, wherein said radio drug is MIBI, being labeled with Tc-99m, allowing label and control of ECD, HMPAO, Tetraphosmine, and MAG3, changing a partition system allowing control of the radio-drugs.
